(19) United States
(12) Patent Application Publication
(10) Pub. No.: US 2004/0249205 A1
(43) Pub. Date: Dec. 9, 2004

(54) Process for producing hydroxyamino acid derivative

(76) Inventors: Naoki Matsumoto, Yokohama-shi (JP); Katsura Kaneko, Yokohama-shi (JP); Hazuki Nagai, Fujisawa-shi (JP); Kaname Konuki, Iwata-shi (JP); Toshio Tsuchida, Yamato-shi (JP); Kunio Issiki, Zama-shi (JP)

Correspondence Address: JACOBSON HOLMAN PLLC, 400 SEVENTH STREET N.W., SUITE 600, WASHINGTON, DC, 20004, US

(21) Appl. No.: 10/482,119

(22) PCT Filed: Jun. 26, 2002
(86) PCT No.: PCT/JP02/06414

(51) Int. Cl. C07C 229/02

(52) U.S. Cl. 562/567

(57) ABSTRACT

Provided is a method of preparing a hydroxyamino acid derivative, particularly in an optically active form thereof with high efficiency. Specifically, provided is a method of preparing hydroxyamino acid derivatives represented by Formula (I) or salts thereof:

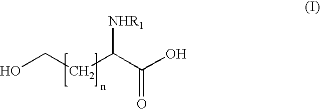

, characterized in that an amino acid derivative represented by Formula (II):

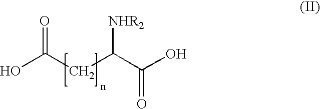

is subjected to a selective reduction reaction of a carboxyl group at a side chain terminus in a solvent, and then, optionally, subjected to an elimination reaction of a protecting group of an amino group or a salt-formation reaction of an amino group or a carboxyl group.

### TECHNICAL FIELD

The present invention relates to a method of preparing a hydroxyamino acid derivative, particularly in an optically active form thereof.

### TECHNICAL BACKGROUND

Hydroxyamino acid derivatives, in particular, the optically active substances, are useful as intermediates for the preparation of pharmaceuticals. For example, optically active ε-hydroxynorleucine is employed as an intermediate for the preparation of Omapatrilat (BMS-186716), which is an antihypertensive based on a new mechanism, that is, a dual inhibition of an angiotensin converting enzyme (ACE) and neutral endopeptidase (NEP).

On the other hand, many approaches to the method of preparing hydroxyamino acid derivatives, and particularly with the optically active form, have been proposed so far. For example, (1) Tetrahedron, 44, 2633 (1988), (2) Tetrahedron Lett., 39, 5671 (1998), and (3) Tetrahedron Lett., 36, 439 (1995) disclose a method of preparing ε-hydroxynorleucine (including the protected one) from L-lysin; (4) Japanese Unexamined Patent Publication (KOKAI) Heisei No. 7-48259 discloses a method of preparing ε-hydroxynorleucine by an optical kinetic resolution of racemic N-acetyl-ε-hydroxynorleucine, synthesized from a starting material of diethyl malonate, with pig liver acylase; and (5) Can. J. Res. Sect., B26, 387 (1948) and (6) J. Med. Chem., 21, 1030 (1978) disclose a method of an enzymatic optical resolution of racemic ε-hydroxynorleucine prepared from a starting material of cyclohexene.

In addition, (7) Tetrahedron Asymmetry, 11, 991-994 (2000) discloses a method in which L-glutamic acid is used as a starting material, its amino group and carboxyl group are pre-protected together as a borooxazoline, and a carboxyl group at a side chain terminus is reduced to prepare δ-hydroxynorvaline; (8) J. Chem. Soc., Chem. Commun., 1583-1684 (1987) discloses a method in which an amino group of L-glutamic acid methylester is protected by a trityl group, after which a side chain terminal methoxycarbonyl group is reduced using lithium aluminum hydride to prepare optically active δ-hydroxynorvaline; and (9) Denki kagaku oyobi kogyo butsuri kagaku, 52, 165 (1987) discloses a method in which a γ-hydrazine derivative of L-glutamic acid is reduced using an electrode to prepare optically active δ-hydroxynorvaline.

However, the preparation methods according to (1) to (3) require multi-step reaction processes. All of the preparation methods according to (4) to (6) produce undesired enantiomeric isomers in half amounts, making it problematic. In addition, in the preparation method according to (7), an agent used in a reduction reaction is expensive; and the preparation method according to (8) has a drawback because a carboxyl group to be reduced must be pre-esterified and purified prior to the reduction, resulting in increase of reaction processes. The preparation method according to (9) requires a special device for an electrode reaction. These methods are insufficient.

### DISCLOSURE OF THE INVENTION

Accordingly, it is an object of the present invention to provide a method of preparing a hydroxyamino acid derivative represented by Formula (I) below, particularly in an optically active form thereof with high efficiency.

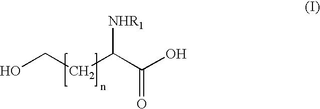

The present inventors conducted extensive research to solve the aforementioned problem. They discovered that the hydroxyamino acid derivative represented by Formula (I) can be prepared efficiently by the steps of: pre-protecting an amino group of an α-amino acid derivative represented by Formula (III) below, which can be prepared in large amounts by a fermentation method and has a carboxyl group at a side chain terminus thereof; and subjecting it to a reduction reaction under certain conditions to selectively reduce carboxyl groups at side chain termini thereof. The present invention is based on such knowledge.

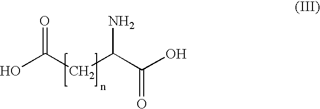

That is, the present invention provides a method of preparing hydroxyamino acid derivatives represented by Formula (I) or their salts:

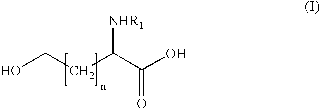

(wherein R₁ represents a hydrogen atom or a protecting group of an amino group, and n represents an integer that is either 2 or 3), characterized in that

an amino acid derivative represented by Formula (II):

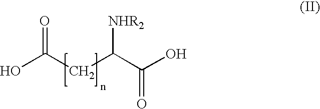

(wherein R₂ represents a protecting group of an amino group and n is defined as in Formula (I))

is subjected to a selective reduction reaction of a carboxyl group at a side chain terminus in a solvent, and then, optionally, subjected to an elimination reaction of a protecting group of an amino group or a salt-formation reaction of an amino group or a carboxyl group.

### BEST MODES FOR CARRYING OUT THE INVENTION

A compound of Formula (II), that is a starting compound in the preparation method of the present invention, is prepared by introducing a protecting group into an amino group of α-amino acid represented by Formula (III) above. As a protecting group of an amino group, known protecting groups of an amino group described in, for example, “Protecting Groups in Organic Chemistry,” John Wiley and Sons, 1991, can be employed without any limitation. Specific examples of such protecting groups include carbamate-based protecting groups such as a tert-butoxycarbonyl group, benzyloxycarbonyl group, and alkoxycarbonyl group; acyl-based protecting groups such as a formyl group, acetyl group, and propionyl group; silyl-based protecting groups such as a trimethylsilyl group, triethylsilyl group, tert-butyldimethylsilyl group, and tert-butyldiphenylsilyl group; and sulfonamide-based protecting groups such as a methanesulfonyl group, benzenesulfonyl group, and p-toluenesulfonyl group.

The protecting group can be introduced via a known method, for example, by causing a protecting agent, such as dibutyl carbonate, tert-butyloxycarbonyl chloride, benzyloxycarbonyl chloride, acetyl chloride, trimethylsilyl chloride, or tert-butyldimethylsilyl chloride, to react thereto in a presence of a base such as sodium hydroxide, potassium hydroxide, triethylamine, or imidazole in water or in an appropriate organic solvent at a temperature ranging from −20° C. to the reflux temperature of the solvent.

A carboxyl group at a side chain terminus of the compound of Formula (II) thus obtained can be selectively reduced in an appropriate solvent with a process according to any of (a), (b), or (c) below to obtain a compound of Formula (I) in which R₁ is a protecting group of an amino group.

Process (a) is a process of subjecting a carboxyl group at a side chain terminus to a reaction with an activating agent to convert it into an active ester group, and then reducing the active ester group obtained to a hydroxymethyl group by metallic borohydride.

In this process, first the compound of Formula (II) is converted to an active ester derivative by subjecting it to a reaction with an activating agent such as carbonyldiimidazole, N-hydroxysuccinimide, N-hydroxybenzotriazole, thionyl chloride, sulfuryl chloride, phosphorous oxychloride, or phosphorus pentachloride in an appropriate inert organic solvent (such as tetrahydrofuran, diethyl ether, 1,4-dioxane, hexane, toluene, benzene, methylene chloride, chloroform, acetonitrile, or dimethylformamide), optionally in the presence of a base such as pyridine or triethylamine or a condensing agent such as dicyclohexylcarbodiimide or 1-ethyl-3-(3-dimethylaminopropyl) carbodiimide.

The reaction temperature ranges from −78° C. to the reflux temperature of the solvent, preferably from −30° C. to 50° C. The reaction time ranges from 10 minutes to one night, preferably from 1 hour to 3 hours. Preferably, the molar ratio of the compound of Formula (II) to an activating agent employed ranges from 1:1 to 1:10, and further preferably from 1:1 to 1:2. The molar ratio of the compound of Formula (II) to a condensing agent employed preferably ranges from 1:1 to 1:3.

The active ester derivative thus obtained, usually without isolation, can be subjected to a reaction with metallic borohydride such as sodium borohydride or sodium triacetoxyborohydride to obtain a hydroxyamino acid derivative represented by Formula (I) in which R₁ is a protecting group of an amino group. The reaction temperature may range from −78° C. to the reflux temperature of the solvent, and preferably from −30° C. to 50° C. The reaction time usually ranges from 10 minutes to one night, preferably from 30 minutes to 120 minutes. Preferably, the molar ratio of the compound of Formula (II) to the metallic borohydride employed ranges from 1:1 to 1:10, and further preferably from 1:1 to 1:5.

Process (b) is a reduction process of a carboxyl group at a side chain terminus to a hydroxymethyl group with borane or a borane-ether complex.

In this process, the compound of Formula (II) is subjected to a reaction with borane or a borane-ether complex (such as borane-tetrahydrofuran complex) in an appropriate inert organic solvent (such as tetrahydrofuran, diethyl ether, 1,4-dioxane, hexane, toluene, benzene, methylene chloride, chloroform, acetonitrile, or dimethylformamide) to obtain a hydroxyamino acid derivative represented by Formula (I) in which R₁ is a protecting group of an amino group. The reaction temperature may range from −78° C. to the reflux temperature of the solvent, and preferably from −20° C. to room temperature. The reaction time usually ranges from 10 minutes to 6 hours, and preferably from 20 minutes to 120 minutes. Preferably, the molar ratio of the compound of Formula (II) to borane or a borane-ether complex employed ranges from 1:1 to 1:10, and further preferably from 1:1 to 1:2.

Process (c) is a reduction process of a carboxyl group at a side chain terminus with metallic borohydride and at least one compound selected from the group consisting of protonic acid, Lewis acid, dialkylsulfuric acid, iodine, and alkyl iodide to convert it into a hydroxymethyl group.

In this process, the compound of Formula (II) is subjected to a reaction with at least one compound selected from the group consisting of protonic acid (such as hydrochloric acid, sulfuric acid, acetic acid, trifluoroacetic acid, p-toluenesulfonic acid, methanesulfonic acid, or catechol), Lewis acid (such as a boron trifluoride diethyl ether complex, a boron trifluoride dimethyl ether complex, aluminum chloride, or zinc chloride), dialkylsulfuric acid (such as dimethylsulfuric acid or diethylsulfuric acid), iodine, and alkyl iodide (such as methyl iodide or ethyl iodide) in the presence of metallic borohydride such as sodium borohydride, lithium borohydride, or potassium borohydride in an appropriate inert organic solvent (such as tetrahydrofuran, diethyl ether, 1,4-dioxane, hexane, toluene, benzene, methylene chloride, chloroform, or dimethylformamide) to obtain a hydroxyamino acid derivative represented by Formula (I) in which R₁ is a protecting group of an amino group. The reaction temperature may range from −50° C. to the reflux temperature of the solvent, preferably −20° C. to room temperature. The reaction time usually ranges from 10 minutes to 6 hours, and preferably from 30 minutes to 2 hours. Preferably, the molar ratio of the compound of Formula (II) to the metallic borohydride employed ranges from 1:1 to 1:10, and further preferably from 1:1 to 1:2.

In all (a) to (c) above, the hydroxyamino acid derivative of Formula (I) thus obtained in which R₁ is a protecting group of an amino group can be purified as follows; first, methanol, diluted hydrochloric acid, or the like is added in the reaction mixture to decompose the remaining reducing agent, such as a metallic borohydride or a borane-ether complex, and then the reaction mixture is extracted with an organic solvent such as methylene chloride, ethyl acetate, or toluene, or optionally, it is subjected to a chromatography with silica gel, ion-exchange resin, or the like, or recrystallization in which it is derived to a crystalline salt.

Optionally, the protecting group of the aforementioned hydroxyamino acid derivatives can be eliminated by an elimination reaction that is per se known to obtain a hydroxyamino acid derivative of Formula (I) in which R₁ is a hydrogen atom. Subsequently, it can be optionally subjected to a salt-formation reaction with an amino group or a carboxyl group to convert it into a salt of interest.

### EXAMPLES

The present invention will be explained more specifically through the following specific examples. However, the method of the present invention is not limited to the following examples.

### Example 1

### Preparation of L-N-benzyloxycarbonyl-ε-hydroxynorleucine (1)

100 mg of L-N-benzyloxycarbonyl-homoglutamic acid was dissolved in 5 ml of tetrahydrofuran, and 66 mg of carbonyldiimidazole in 0.1 ml of dimethylformamide was added. After stirring at room temperature for 30 minutes, to the mixture, 39 mg of sodium borohydride was added, followed by further stirring for 1.5 hours. An excess amount of agent was degraded by the addition of methanol, and the solvent was removed under reduced pressure. The residue obtained was dissolved in 5 ml of 1 mol/l sodium hydroxide solution and washed with 20 ml of ethyl acetate. 5 ml of 2 mol/l hydrochloric acid was added to the separated aqueous phase and extracted with 20 ml of ethyl acetate. After washing with a saturated saline solution and drying with anhydrous sodium sulfate, the solvent was removed under reduced pressure to obtain 76.2 mg of a crude product of the compound of interest (with a crude yield of 80%).

### Example 2

### Preparation of L-N-benzyloxycarbonyl-ε-hydroxynorleucine (2)

1 g of L-N-benzyloxycarbonyl-homoglutamic acid was dissolved in 30 ml of tetrahydrofuran. To the mixture, a solution of 0.9 mol/l borane-tetrahydrofuran complex in 10 ml tetrahydrofuran was added dropwise while the mixture was cooled on ice, after which stirring was continued for 3 hours. Methanol was added to the mixture until an excess amount of agent was degraded, and the solvent was removed under reduced pressure. The residue obtained was dissolved in 25 ml of 1 mol sodium hydroxide solution and washed with 100 ml of ethyl acetate. 25 ml of 2 mol/l hydrochloric acid was added to the separated aqueous phase and the solution was extracted with 150 ml of ethyl acetate. After washing with a saturated saline solution and drying with anhydrous sodium-sulfate, the solvent was removed under reduced pressure to give 730.3 mg of a crude product of the compound of interest (with a crude yield of 77%).

### Example 3

### Preparation of L-N-tert-butoxycarbonyl-ε-hydroxynorleucine (1)

500 mg of L-N-tert-butoxycarbonyl-homoglutamic acid and 217 mg of sodium borohydride were dissolved in 20 ml of tetrahydrofuran with stirring. To the mixture, 632 mg of iodine in 5 ml tetrahydrofuran solution was added dropwise for 10 minutes while cooling the mixture on ice, and stirring was continued for 1 hour at room temperature. 5 ml of methanol was carefully added to the reaction mixture while cooling the mixture on ice, and stirring was continued for 10 minutes to degrade an excess amount of reducing agent. After removing the solvent under reduced pressure, 15 ml of ethyl acetate was added to the residue, and extracted twice with 15 ml and 5 ml of water the first and second times, respectively. The aqueous phases obtained were combined, and 8 g of sodium chloride was added thereto for saturation. After adjusting to pH 3 with 1 moll of hydrochloric acid, the solution was extracted twice with 20 ml and 10 ml of ethyl acetate the first and second times, respectively. After the organic phases obtained were combined and dried with anhydrous sodium sulfate, they were concentrated under reduced pressure and dried under a vacuum to give 317 mg of a crude product of the compound of interest as a colorless syrup (with a crude yield of 67%).

A portion (184 mg) of the resulting crude product was dissolved in 2.5 ml of 20% methanol and adsorbed to 2 ml of Diaion PA308 ion-exchange resin (manufactured by Mitsubishi Chemical Corporation). After washing with water, it was eluted with 15 ml of 0.1 mol/l hydrochloric acid. The eluate was extracted with ethyl acetate. It was concentrated to give 64 mg of a purified product of the compound of interest. When the specific optical rotation of the purified product obtained was measured, the value corresponded to the value described in the literature (J. Am. Chem. Soc., 104, 3096 (1982)), confirming that configuration had been maintained.

### Example 4

### Preparation of L-N-tert-butoxycarbonyl-ε-hydroxynorleucine (2)

21.7 mg of sodium borohydride and 72.8 μl (0.575 mmol) of boron trifluoride ether complex were sequentially added to 100 mg of L-N-tert-butoxycarbonyl-homoglutamic acid in 1 ml of tetrahydrofuran solution while cooling the solution on ice. After continuing the reaction for 2 hours at the same temperature, 30 ml of water was added to the reaction mixture. Furthermore, the mixture was adjusted to pH 4 with 0.1 mol/l of hydrochloric acid and stirring was continued for 10 minutes. Subsequently, after adjusting the pH to 9 with 5 mol/l of sodium hydroxide solution, the solution was washed with ethyl acetate. The aqueous phase was adjusted to pH 3 with 1 mol/l of hydrochloric acid, and was extracted twice with 30 ml of ethyl acetate both times. The ethyl acetate phase obtained was washed with 0.001 mol/l of hydrochloric acid, and then dried with anhydrous sodium sulfate. The solvent was removed by concentration under reduced pressure to obtain 51.1 mg of a crude product containing the compound of interest as a main component (with a crude yield of 53%).

### Example 5

### Preparation of L-N-tert-benzyloxycarbonyl-ε-hydroxynorleucine (3)

1 g of L-N-benzyloxycarbonyl-homoglutamic acid was dissolved in 20 ml of tetrahydrofuran. 365 mg of sodium borohydride was further added thereto and the resultant was stirred for 10 minutes. To the mixture, 749 mg of iodine in 5 ml tetrahydrofuran solution was added dropwise for 10 minutes while cooling the mixture on ice, and the resultant mixture was stirred for 3 hours at room temperature. 1 ml of methanol was carefully added to the reaction mixture while cooling the mixture on ice. Stirring was continued for 30 minutes to degrade an excess amount of reducing agent. 100 ml of water and 6 mol/l of sodium hydroxide solution were added thereto. After adjusting to pH 8, it was washed with ethyl acetate. Sodium hydroxide was added to the aqueous phase obtained for saturation. After adjusting the pH to 3 with 1 mol/l hydrochloric acid, extraction was carried out twice with 100 ml and 60 ml of ethyl acetate the first and second times, respectively. The organic phases obtained were combined and dried with anhydrous sodium sulfate. Thereafter, the solvent was removed under reduced pressure to give 605 mg of a crude product of the compound of interest (with a crude yield of 64%).

### Example 6

### Preparation of L-N-tert-benzyloxycarbonyl-ε-hydroxynorleucine (4)

500 mg of L-N-benzyloxycarbonyl-homoglutamic acid was dissolved in 10 ml of tetrahydrofuran. 193 mg of sodium borohydride was further added thereto. To the mixture, 0.2 ml of trifluoroacetic acid was added dropwise for 10 minutes while cooling the mixture on ice, and the mixture was stirred overnight at room temperature. After the addition of 1 ml of 0.1 mol/l hydrochloric acid, the solution was stirred for 30 minutes to degrade an excess amount of reducing agent. The mixture was adjusted to pH 8 with 6 mol/l of sodium hydroxide solution, and washed with ethyl acetate. After adjusted to pH 3 with 1 mol/l of hydrochloric acid, the aqueous phase obtained was extracted twice with 50 ml and 30 ml of ethyl acetate the first and second times, respectively. The organic phases obtained were combined and dried with anhydrous sodium sulfate. Thereafter, the solvent was removed under reduced pressure to give 266 mg of a crude product of the compound of interest (with a crude yield of 56%).

The resultant crude product was purified by a preparative TLC (Art. 105744 manufactured by Merck, developing solvent: chloroform/methanol/acetic acid=5:1:0.1), giving 167 mg of the compound of interest was obtained (with a yield of 36%).

### Example 7

### Preparation of L-N-benzyloxycarbonyl-ε-hydroxynorleucine (5)

193 mg of sodium borohydride was dissolved in 5 ml of tetrahydrofuran. 558 mg of catechol in 5 ml of tetrahydrofuran solution and 500 mg of L-N-benzyloxycarbonyl-homoglutamic acid in 3 ml of tetrahydrofuran solution were sequentially added and the resultant mixture was stirred overnight at room temperature. 0.2 ml of 0.1 mol/l hydrochloric acid was carefully added and the resultant mixture was stirred for 30 minutes to degrade an excess amount of reducing agent. The solution was adjusted to pH 8 with 6 mol/l sodium hydroxide solution, and washed with ethyl acetate. After adjusted to pH 3 with 1 mol/l hydrochloric acid, the aqueous phase obtained was extracted with 30 ml of ethyl acetate. The organic phase obtained was dried with anhydrous sodium sulfate, and the solvent was removed under reduced pressure to give 393 mg of a crude product of the compound of interest (with a crude yield of 83%).

The resultant crude product was purified by a preparative TLC (Art. 105744 manufactured by Merck, developing solvent: chloroform/methanol/acetic acid=5:1:0.1), giving 98 mg of the compound of interest was obtained (with a yield of 21%).

### Example 8

### Preparation of L-ε-hydroxynorleucine

10.0 g of L-N-benzyloxycarbonyl-ε-hydroxynorleucine prepared in the method of Example 6 was dissolved in 200 ml of methanol. 1.0 g of 10% Pd/C was added and the mixture was stirred overnight at room temperature and normal pressure under hydrogen gasflow. 500 mg of 10% Pd/C was further added and stirring was continued for further 2 hours in the same manner. After adding 200 ml of water to the reaction solution and filtering with celite, the filtrate was removed under reduced pressure. The resultant white solid was washed by suspending in 50 ml of methanol. Thereafter, crystals were collected by filtration and dried to obtain 4.81 g of the compound of interest (with a yield of 92%).

### Example 9

### Preparation of L-N-benzyloxycarbonyl-δ-hydroxynorvaline

33.6 mg of sodium borohydride was added to 100 mg of L-N-benzyloxycarbonyl-glutamic acid in 2 ml of tetrahydrofuran solution while cooling it on ice. 90.2 mg of iodine in 1 ml of tetrahydrofuran solution was further added dropwise. The temperature of the reaction solution was returned to room temperature and stirring was continued overnight. 20 ml of water was added to the reaction mixture and the solution was washed twice with 20 ml and 20 ml of ethyl acetate the first and second times, respectively. The aqueous phase was adjusted to pH 1.1 with 2 ml of 1 mol/l of hydrochloric acid, and extracted twice with 20 ml and 20 ml of ethyl acetate the first and second times, respectively. The organic phases obtained were combined and dried with anhydrous sodium sulfate. Thereafter, the solvent was removed under reduced pressure to afford 31 mg of a crude product of the product of interest (with a crude yield of 33%).

### Industrial Applicability

In each of the preparation methods of the present invention, the configuration is maintained in each reaction process. It is characterized in that a carboxyl group at a side chain terminus can be selectively reduced alone, without the reduction of a carboxyl group at the a position. Therefore, when the optically active compounds of Formulas (II) and (III) are employed as starting compounds, the corresponding optically active hydroxyamino acid derivative of Formula (I) can be prepared with high efficiency. Thus, according to the present invention, L-hydroxynorvaline or L-hydroxynorleucine, their derivatives in which an amino group is protected, or salts thereof can be economically prepared using L-glutamic acid or L-homoglutamic acid that can be prepared in a large scale by fermentation.

1. A method of preparing hydroxyamino acid derivatives represented by formula (i) or salts thereof: 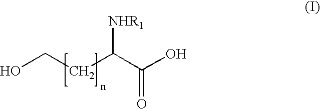
(wherein R₁ represents a hydrogen atom or a protecting group of an amino group, and n represents an integer that is either 2 or 3), characterized in that
an amino acid derivative represented by Formula (II): 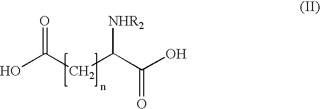
(wherein R₂ represents a protecting group of an amino group and n is defined as in Formula (I) )
is subjected to a selective reduction reaction of a carboxyl group at a side chain terminus in a solvent, and then, optionally, subjected to an elimination reaction of a protecting group of an amino group or a salt-formation reaction of an amino group or a carboxyl group.

2. The method of preparing according to claim 1, wherein said protecting group of an amino group is a carbamate-based protecting group, an acyl-based a protecting group, a silyl-based protecting group, or a sulfonamide-based protecting group.

3. The method of preparing according to claim 1, wherein said solvent is an inert organic solvent.

4. The method of preparing according to claim 1, wherein the selective reduction reaction of a carboxyl group at a side chain terminus is a reaction comprising a process of subjecting a carboxyl group at a side chain terminus to a reaction with an activating agent to convert the carboxyl group into an active ester group, and then reducing the active ester group obtained with metallic borohydride to convert the active ester group into a hydroxymethyl group (process (a)).

5. The method of preparing according to claim 4, wherein the reaction with an activating agent in the process (a) is carried out in the presence of a condensing agent.

6. The method of preparing according to claim 4, wherein said activating agent is carbonyldiimidazole, N-hydroxysuccinimide, N-hydroxybenzotriazole, thionyl chloride, sulfuryl chloride, phosphorous oxychloride, or phosphorus pentachloride.

7. The method of preparing according to claim 1, wherein the selective reduction reaction of a carboxyl group at a side chain terminus is a reaction comprising a process of reducing a carboxyl group at a side chain terminus with borane or a borane-ether complex to convert the carboxyl group into a hydroxymethyl group (process (b)).

8. The method of preparing according to claim 1, wherein the selective reduction reaction of a carboxyl group at a side chain terminus is a reaction comprising a process of reducing a side chain carboxyl group with a metallic borohydride and at least one compound selected from the group consisting of protonic acid, Lewis acid, dialkylsulfuric acid, iodine, and alkyl iodide to convert the side chain carboxyl group into a hydroxymethyl group (process (c)).
